(19) United States
(12) Patent Application Publication
(10) Pub. No.: US 2018/0360334 A1
(43) Pub. Date: Dec. 20, 2018

(54) SYSTEMS AND METHODS FOR AUTOMATICALLY PLOTTING ELECTROCARDIOGRAM GRIDS ON A DISPLAY

(71) Applicant: UBER DIAGNOSTICS PTE. LTD., Singapore (SG)

(72) Inventors: Ashim Roy, Bangalore (IN); Avin Agarwal, Bangalore (IN)

(21) Appl. No.: 16/109,738

(22) Filed: Aug. 22, 2018

Related U.S. Application Data
(63) Continuation of application No. PCT/IB2017/050990, filed on Feb. 22, 2017.

(30) Foreign Application Priority Data
Feb. 22, 2016 (IN) 201621005977

(51) Int. Cl. A61B 5/044; A61B 5/0408; G16H 50/20

(52) U.S. Cl. CPC A61B 5/044; A61B 5/04012; G16H 50/20; A61B 5/04085

(57) ABSTRACT

Computerized systems for automatically plotting electrocardiogram on a display device. The device may be polled to determine the configuration of the display, such as a screen height, length, resolution, etc. The display is divided into axes of a grid at particular spatial intervals fit to the display to form a dynamically-oriented background grid. The ECG signal is then plotted with data points corresponding in real time to the signal on the background grid to plot the ECG signal on the background grid, wherein the dividing and the synchronizing dynamically repositioning each grid line and each incoming data point based on a configuration of the display.

### RELATED APPLICATIONS

This application claims priority under 35 U.S.C. § 120 to, and is a continuation of, co-pending International Application PCT/IB2017/050990, filed Feb. 22, 2017 and designating the US, which claims priority to Indian Application 201621005977, filed Feb. 22, 2016, such Indian Application also being claimed priority to under 35 U.S.C. § 119. These Indian and International applications are incorporated by reference herein in their entireties.

### BACKGROUND

The heart is a muscular organ in humans and other animals that pumps blood through the blood vessels of the circulatory system. As shown in FIG. 2, in humans, other mammals, and birds, heart 4 is divided into four chambers: upper left and right atria; and lower left and right ventricles. A cardiac cycle refers to a complete heartbeat from its generation to the beginning of the next beat, and so includes the diastole, the systole, and the intervening pause. The frequency of the cardiac cycle is described by the heart rate, which is typically expressed as beats per minute. A normal rhythmical heartbeat, called a sinus rhythm, is established by the sinoatrial node, the heart's pacemaker. An electrical signal is created that travels through the heart 4, causing the heart muscle to contract. Each electrical signal begins in a group of cells called the sinus node or sino-atrial (SA) node 41. As shown in FIG. 2A, SA node 41 is located in the right atrium, which is the upper right chamber of the heart. In a healthy adult heart 4 at rest, the SA node 41 sends an electrical signal to begin a new heartbeat 60 to 100 times a minute.

As shown in FIG. 1, electrocardiography (ECG or EKG, herein referred to as ECG) 1 is the process of recording the electrical activity of a heart over a period of time using electrodes 5 placed on a patient's body. Electrodes 5 detect tiny electrical changes on the skin that arise from the heart muscle depolarizing during each heartbeat. In FIG. 1, a patient's heart activity is being recorded using electrocardiogram. As electrical changes progress from the SA node 41 (FIG. 2A), the signal travels through the right and left atria. This causes the atria to contract, which helps move blood into the heart's lower chambers, the ventricles. The electrical signal moving through the atria is recorded as P-wave on the machine called an electrocardiogram (also ECG or EKG, herein referred to as EKG) 2 performing electrocardiography. The electrical signals are picked up by electrodes 5 and delivered to EKG 2 via electrical connections 6.

FIG. 3 is a detailed illustration of a typical ECG 1 showing a repeating cycle of three electrical entities: a P-wave (atrial depolarization), a QRS complex (ventricular depolarization), and a T-wave (ventricular repolarization). The ECG 1 is traditionally interpreted methodically to catch important findings. As the electrical signal passes through the heart, it moves between the atria and ventricles through a group of cells called the atrio-ventricular (AV) node 42 (FIG. 2A). The signal slows down as it passes through AV node 42. This slowing allows the ventricles enough time to finish filling with blood. On ECG 1, this part of the process is the flat line between the end of the P-wave and the beginning of the Q-wave. The electrical signal then leaves the AV node 42 and travels along a pathway called the bundle of His 43. From there, the signal travels into the right and left bundle branches. The signal spreads quickly across the heart's ventricles, causing them to contract and pump blood to the lungs and the rest of the body. This process is recorded as the QRS waves on the ECG 1. The ventricles then recover their normal electrical state (shown as the T-wave on ECG 1). The muscle stops contracting to allow the heart to refill with blood. This entire process continues over and over with each new heartbeat.

In ECG 1, the P-wave represents depolarization of the atria. Atrial depolarization spreads from the SA node 41 towards the AV node 42, and from the right atrium to the left atrium. The P-wave is typically upright in most leads except for a VR; an unusual P-wave axis (inverted in other leads) can indicate an ectopic atrial pacemaker. If the P-wave is of unusually long duration, it may represent atrial enlargement. Typically, a large right atrium gives a tall, peaked p-wave while a large left atrium gives a two-humped bifid P-wave. The P-wave duration is less than 80 ms.

In ECG 1, the PR interval is measured from the beginning of the P-wave to the beginning of the QRS complex. This interval reflects the time the electrical impulse takes to travel from the sinus node through the AV node 42. A PR interval shorter than 120 ms suggests that the electrical impulse is bypassing the AV node 42 as in Wolf-Parkinson-White syndrome. A PR interval consistently longer than 200 ms diagnoses first degree atrioventricular block. The PR segment (the portion of the tracing after the P-wave and before the QRS complex) is typically completely flat but may be depressed in pericarditis. The PR interval is 120 to 200 ms.

In ECG 1, the QRS complex represents the rapid depolarization of the right and left ventricles. The ventricles have a large muscle mass compared to the atria, so the QRS complex usually has a much larger amplitude than the P-wave. If the QRS complex is wide (longer than 120 ms) it suggests disruption of the heart's conduction system, such as in LBBB, RBBB, or ventricular rhythms such as ventricular tachycardia. Metabolic issues such as severe hyperkalemia, or TCA overdose can also widen the QRS complex. An unusually tall QRS complex may represent left ventricular hypertrophy while a very low-amplitude QRS complex may represent a pericardial effusion or infiltrative myocardial disease. The QRS interval is 80 to 100 ms.

In ECG 1, the J-point is the point at which the QRS complex finishes and the ST segment begins. The J point may be elevated as a normal variant. The appearance of a separate J wave or Osborn wave at the J point is pathognomonic of hypothermia or hypercalcemia. The J point may be elevated as a normal variant. The appearance of a separate J wave or Osborn wave at the J point is pathognomonic of hypothermia or hypercalcemia.

In ECG 1, the ST segment connects the QRS complex and the T-wave; it represents the period when the ventricles are depolarized. It is usually isoelectric but may be depressed or elevated with myocardial infarction or ischemia. ST depression can also be caused by LVH or digoxin. ST elevation can also be caused by pericarditis, Brugada syndrome, or can be a normal variant (J-point elevation). The ST segment interval is 160 ms.

In ECG 1, the T-wave represents the repolarization of the ventricles. It is generally upright in all leads except a VR and lead V1. Inverted T-waves can be a sign of myocardial ischemia, LVH, high intracranial pressure, or metabolic abnormalities. Peaked T-waves can be a sign of hyperkalemia or very early myocardial infarction.

In ECG 1, the QT interval is measured from the beginning of the QRS complex to the end of the T-wave. Acceptable ranges vary with heart rate, so it must be corrected by dividing by the square root of the distance between successive R peaks (RR interval). A prolonged QT interval is a risk factor for ventricular tachyarrhythmias and sudden death. Long QT can arise as a genetic syndrome, or as a side effect of certain medications. An unusually short QT can be seen in severe hypercalcemia. The QT interval is less than 440 ms.

In an ECG, a U wave is hypothesized to be caused by the repolarization of the interventricular septum. It normally has a low amplitude, and even more often is completely absent. If the U wave is very prominent, suspect hypokalemia, hypercalcemia or hyperthyroidism.

The various signals discussed above and parameters of the same are typically visually plotted on ECG machine 2. Twelve channels, each corresponding to a single lead, may be visible on a single display, such as a screen or sheet of paper, with a background grid for easy determination of relative signal parameters.

### SUMMARY

Example embodiments include computerized systems for automatically plotting electrocardiogram on a display device. The device may be polled to determine the configuration of the display, such as a screen height, length, resolution, etc. The display is divided into axes of a grid at particular spatial intervals fit to the display to form a dynamically-oriented background grid. The ECG signal is then plotted with data points corresponding in real time to the signal on the background grid to plot the ECG signal on the background grid, wherein the dividing and the synchronizing dynamically repositioning each grid line and each incoming data point based on a configuration of the display.

### BRIEF DESCRIPTIONS OF THE DRAWINGS

Example embodiments will become more apparent by describing, in detail, the attached drawings, wherein like elements are represented by like reference numerals, which are given by way of illustration only and thus do not limit the example embodiments herein.

FIG. 1 is an illustration of a related art electrocardiogram system.

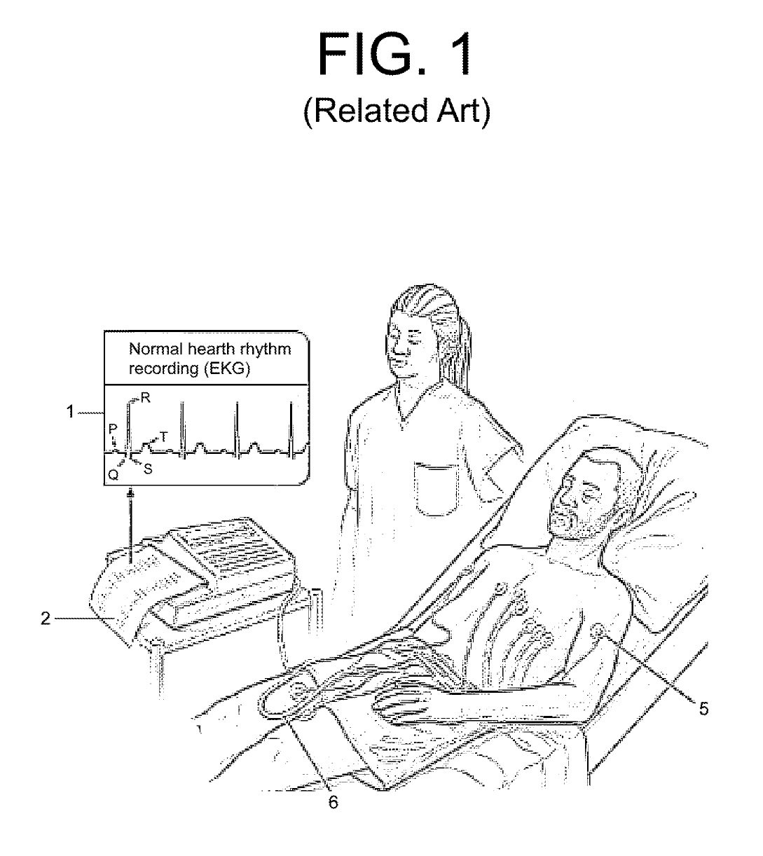

FIG. 2 is an illustration of a human heart electrical system.

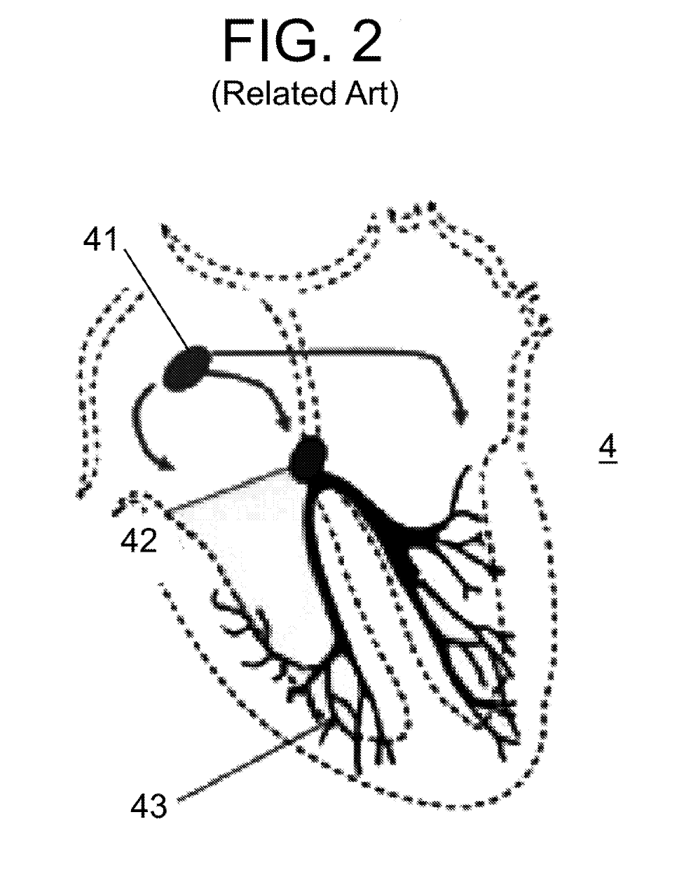

FIG. 3 is an illustration of an ECG of a heartbeat electrical signal.

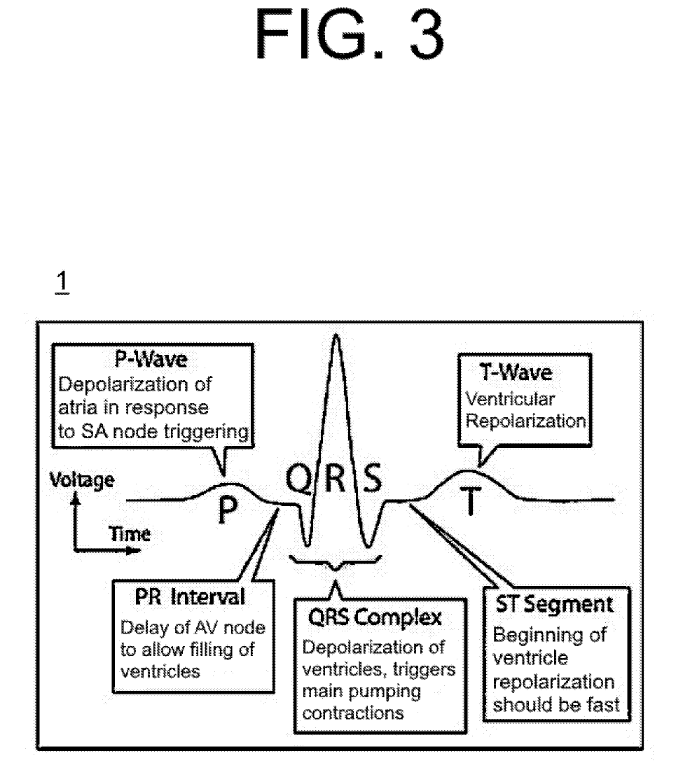

FIG. 4 is an illustration of a schematic of an example embodiment EKG system.

FIG. 5 is an illustration of an output display of an ECG signal in an example method.

### DETAILED DESCRIPTION

Because this is a patent document, general broad rules of construction should be applied when reading it. Everything described and shown in this document is an example of subject matter falling within the scope of the claims, appended below. Any specific structural and functional details disclosed herein are merely for purposes of describing how to make and use examples. Several different embodiments and methods not specifically disclosed herein may fall within the claim scope; as such, the claims may be embodied in many alternate forms and should not be construed as limited to only examples set forth herein.

It will be understood that, although the terms first, second, etc. may be used herein to describe various elements, these elements should not be limited to any order by these terms. These terms are used only to distinguish one element from another; where there are “second” or higher ordinals, there merely must be that many number of elements, without necessarily any difference or other relationship. For example, a first element could be termed a second element, and, similarly, a second element could be termed a first element, without departing from the scope of example embodiments or methods. As used herein, the term “and/or” includes all combinations of one or more of the associated listed items. The use of “etc.” is defined as “et cetera” and indicates the inclusion of all other elements belonging to the same group of the preceding items, in any “and/or” combination(s).

It will be understood that when an element is referred to as being “connected,” “coupled,” “mated,” “attached,” “fixed,” etc. to another element, it can be directly connected to the other element, or intervening elements may be present. In contrast, when an element is referred to as being “directly connected,” “directly coupled,” etc. to another element, there are no intervening elements present. Other words used to describe the relationship between elements should be interpreted in a like fashion (e.g., “between” versus “directly between,” “adjacent” versus “directly adjacent,” etc.).

As used herein, the singular forms “a,” “an,” and “the” are intended to include both the singular and plural forms, unless the language explicitly indicates otherwise. It will be further understood that the terms “comprises,” “comprising,” “includes,” and/or “including,” when used herein, specify the presence of stated features, characteristics, steps, operations, elements, and/or components, but do not themselves preclude the presence or addition of one or more other features, characteristics, steps, operations, elements, components, and/or groups thereof. The use of “about” in connection with values indicates effective approximation, and such values may vary within a range having substantially similar activity or functionality. As such, values referred to as “about” include similar values and precisions expected with applicable manufacturing tolerances and unavoidable impurities in the element of the value, and generally would be expected to vary less than 15% of the value itself.

The structures and operations discussed below may occur out of the order described and/or noted in the figures. For example, two operations and/or figures shown in succession may in fact be executed concurrently or may sometimes be executed in the reverse order, depending upon the functionality/acts involved. Similarly, individual operations within example methods described below may be executed repetitively, individually or sequentially, so as to provide looping or other series of operations aside from single operations described below. It should be presumed that any embodiment or method having features and functionality described below, in any workable combination, falls within the scope of example embodiments.

The Inventor has recognized a need for plotting ECG signals on mobile devices and other outputs with smaller screens, potentially of varying sizes and resolutions. Because the plot may include up to twelve distinct signals from as many leads, each signal needs to be identifiably and readably presented on a same sheet or display. Moreover, these signals need to be synchronized with a same background grid for relative comparison and interpretation of medical condition. However, automatically and quickly displaying all this ECG signal information on a variety of different outputs is extremely difficult given the number of different size displays and outputs among mobile and other devices. The Inventor has developed example embodiments and methods described below to address these and other problems recognized by the Inventor with unique solutions enabled by example embodiments.

The present invention is systems and methods of plotting ECG signals on variable configuration devices. In contrast to the present invention, the few example embodiments and example methods discussed below illustrate just a subset of the variety of different configurations that can be used as and/or in connection with the present invention.

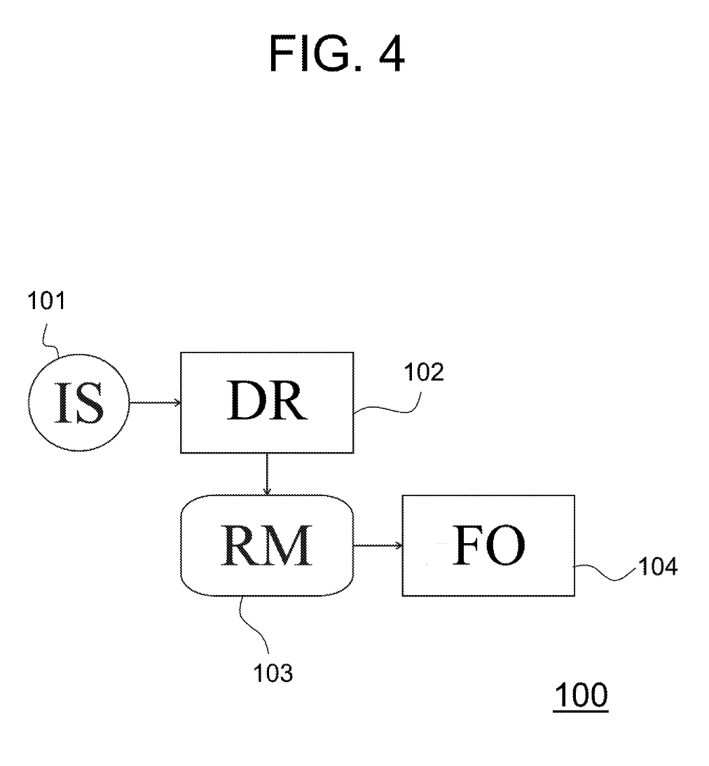

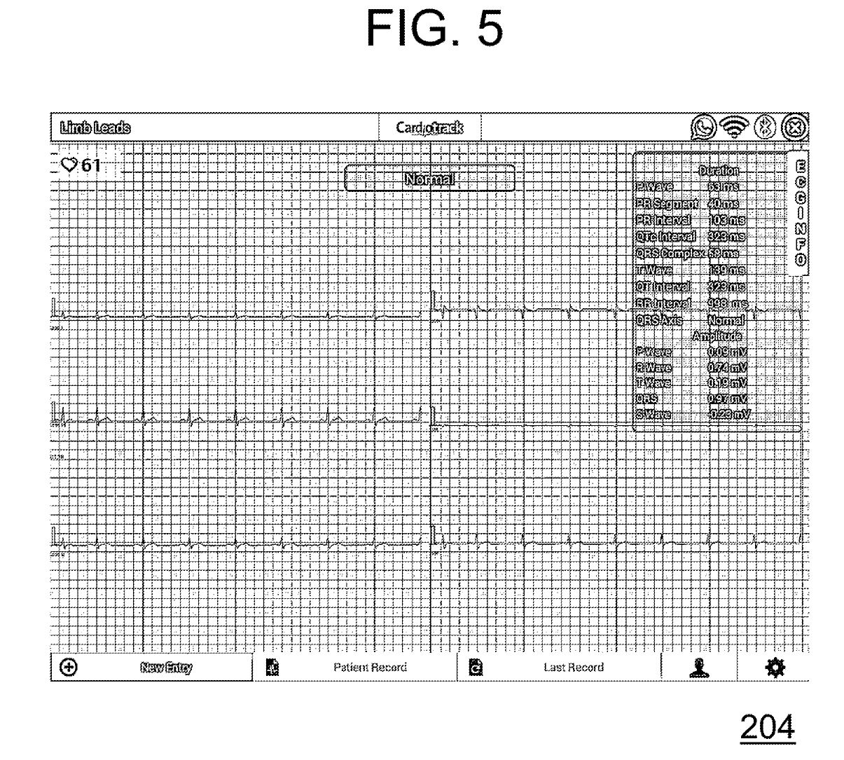

FIG. 4 is an illustration of a schematic of an example embodiment EKG system 100 that is computer processor-driven to receive incoming signals (IS) 101 as per a defined set of display rules (DR) 102 with the display parameters to be processed. A repositioning module (RM) 103 dynamically repositions aspects of the display rules 102 in consonance with IS 101 so that synchronization and viewability is maintained. The computer processor is configured to process the plotting and synchronization so as to display a final output (FO) 104 for the user. For example, FO 104 may be plot 204 of FIG. 5 including a plotted ECG signal on a formed background grid on a display device.

The computer processor may be configured to plot by dividing axes of a grid into pre-defined spatial intervals. For example, axes of an X-axis with 1 second being 25 mm and a Y-axis with 1 mV being 10 mm may be used. To plot, the processor may first poll screen size from a display device to determine screen width and screen height. Then the processor may plot a first set of lines of a pre-defined or desired background color at pre-defined or desired intervals, each set of lines may have a distinct and/or desired thickness. The lines may be straight lines and plotted pixel-by-pixel. For example, these lines may be plotted at a distance of 1 mm each to plot an X-axis in a light pink color.

Next the processor may plot a second set of lines, also at pre-defined or desired intervals, with a distinct thickness. For example, the second set of lines may be a set of lines formed by every fifth line of the first set, with a thickness increased by a factor of 2 to mark 200 millisecond intervals for better readability.

The computer processor may plot a third set of lines, also at pre-defined or desired intervals with a distinct thickness. For example, the third set of lines may be a set of lines formed by every 25th line of the first set, with a thickness increased by a factor of 4 to mark 1 second intervals for better readability.

These pollings and line drawings may be repeated from a defined point of origin, in terms of length, on a display device up to a polled and defined end point, in terms of length on a display device. The lines may be drawn from a polled and defined point of origin, in terms of height, on the display device up to a polled and defined end point, in terms of height, on the display device. This may include plotting vertical lines from an origin at (0,0) to a polled height and width at (length of the display screen, height of the display screen).

A fifth set of lines of a pre-defined or desired color may further be plotted by the processor, pixel by pixel, at a pre-defined distance, to plot the Y-axis on the display device. For example, the fifth set of lines may be straight lines of a light pink color with a pre-defined distance of 1 mm. The computer processor may plot a sixth set of lines, at desired or pre-defined intervals, with a distinct thickness. For example, the sixth set of lines may be formed at every fifth line of the fifth set with thickness that is increased by a factor of 2 to mark 5 milli Volt interval.

The fifth and sixth set of lines for the Y axis may be redrawn from a polled and defined point of origin, in terms of length, on a display device up to a polled and defined end point, in terms of length, on the display device. The lines may be drawn from a polled and defined point of origin, in terms of height, on the display device up to a polled and defined end point, in terms of height, on the display device. This may include plotting horizontal lines from an origin at (0,0) to a polled height and width at (length of the display screen, height of the display screen). Through these sets of lines, grid lines may be formed on a particular display device.

By plotting grid lines pixel-wise, a dynamic grid may be achieved based on the screen size and pixel resolution of the screen, such as on a mobile device. If the resolution or the pixel density is low, fewer lines will be visible on the display, and if it is high, more lines can be plotted for the same size display. The same visibility rule may be applied for screens with different screen sizes but same pixel density or resolution.

The computer processor may synchronize in the real time, the ECG signal display with the formed background grid. The processor may plot the ECG signal on the formed grid of the display device, pixel-by-pixel for each channel of the signal. Then the processor adjusts at least one axis of ECG signal plotting, in real time, over the formed grid. Typically, this axis is the X-axis. The grid may be a dynamically adjusted grid based on device resolution. As the background grid is dynamically adjusted based on device resolution, X axis synchronization of ECG signal bay be achieved by plotting ECG signal samples in real time over the dynamic grid.

The computer processor may vary a sampling rate while plotting the ECG signal. The rate may be varied in correlation to the size and pixel density of the display to achieve desired or required resolution. Increasing or decreasing the sampling rate improves or deteriorates the resolution of ECG signal and is independent of ECG X axis synchronization with the background grid.

The computer processor may further space apart each incoming signal along an axis, the spaced apart distance being correlated with the polled height of display device and number of channels that make up the ECG signal. Typically, this axis is the Y axis. For example, to place each signal at an appropriate distance from each other on Y-axis, the height of the display device may be divided into a layout of 6×2 with six channels of ECG signals plotted in each half.

The computer processor may then dynamically adjust a baseline of the ECG signal being plotted along an axis. Typically, this axis is the Y axis. This adjustment may be done dynamically by identifying the current value of the baseline for any channel and removing the gap between the desired Y-axis position and current Y-axis position. Dynamically adjusting Y-axis may eliminate or lessen abrupt baseline drifts during the course of the scan, and the improved placement of each ECG signal is ensured irrespective of device resolution and display size. The repositioning may be configured dynamically each time there is an input signal to be plotted. The repositioning may be executed batch-wise with assignors to position on a grid for each incoming signal.

FIG. 5 is an illustration of an example embodiment display 204 forming a background grid on any display device with any size or resolution and plotting a real time ECG signal along with formed background grid. Pan and zoom features may be used in FIG. 5 to view the ECG signal on the grid, with x and y synchronization between the grid and ECG signal.

It will be appreciated by one skilled in the art that example embodiments may be varied through routine experimentation and without further inventive activity. For example, although specific types of catheters and visual displays are used in some example embodiments, it is understood that other catheters or venous instrumentation, and other outputs, such as auditory alerts or tactile feedback, can be used in functionally equivalent procedures. Variations are not to be regarded as departure from the spirit and scope of the exemplary embodiments, and all such modifications as would be obvious to one skilled in the art are intended to be included within the scope of the following claims.

What is claimed is:

1. A method for automatically plotting electrocardiogram electrocardiograms on devices of varying configurations, the method comprising:
dividing axes of a grid into spatial intervals of grid lines, with a computer processor, to form a dynamically oriented background grid corresponding to a display;
synchronizing, with the computer processor, data points corresponding to a real time incoming electrocardiogram (ECG) signal with the background grid to plot the ECG signal on the background grid, wherein the dividing and the synchronizing dynamically repositioning each grid line and each incoming data point based on a configuration of the display.

2. The method of claim 1 further comprising:
polling a device including the display for the configuration of the display, wherein the configuration includes screen dimensions of the display.

3. The method of claim 1, wherein the dividing includes plotting a first set of lines of a first color at first intervals and of a first thickness.

4. The method of claim 3, wherein the dividing includes plotting a second set of lines at second intervals and of a second thickness.

5. The method of claim 4, wherein the dividing includes plotting a third set of lines at third intervals and of a third thickness.

6. The method of claim 5, wherein the dividing includes repeating the plotting of the first set of lines, the second set of lines, and the third set of lines from a polled and defined origin in terms of length on the display to a polled and defined endpoint in terms of length on the display.

7. The method of claim 6, wherein the dividing includes plotting a fifth set of lines of a second color, pixel by pixel at a first distance to plot a Y-axis on the display.

8. The method of claim 1, wherein the data points are plotted on the background grid.

9. The method of claim 8, wherein the dividing includes plotting a fifth set of lines of a second color, pixel by pixel at a first distance to plot a Y-axis on the display, and wherein the synchronization includes repeating the plotting the fifth set of lines and the data points from a polled and defined origin in terms of length on the display to a polled and defined end in terms of length on the display.

10. The method of claim 1, wherein the grid lines are straight lines.

11. The method of claim 1, wherein the data points are plotted on the background grid pixel by pixel.

12. The method of claim 1 further comprising:
polling a device including the display for the configuration of the display, wherein the configuration includes screen height and length of the display, wherein the dividing includes,
plotting a first set of lines of a first color at first intervals and of a first thickness,
plotting a second set of lines at second intervals and of a second thickness, and
plotting a third set of lines at third intervals and of a third thickness, and wherein the synchronization includes repeating the plotting the first set of lines, the second set of lines, and the third set of lines from a polled origin in terms of the length and the height on the display to a polled end in terms of the length and the height on the display, so as to plot vertical lines from the origin to the height and the length of the display.

13. The method of claim 1, wherein the dividing includes,
plotting a fifth set of lines of a fifth colour, pixel by pixel, at a fifth distance to plot the Y-axis for the display device, and
plotting the data points on the background grid pixel by pixel, and wherein the synchronization includes repeating the plotting the fifth et of lines and the data points from a polled origin in terms of the length and the height on the display to a polled end in terms of the length and the height on the display, so as to plot horizontal lines from the origin to the height and the length of the display.

14. The method of claim 1, wherein the synchronizing includes plotting the ECG signal on the formed grid on the display.

15. The method of claim 1, wherein the synchronizing includes adjusting at least one axis of the ECG signal in real time over the formed grid.

16. The method of claim 1, wherein the synchronization includes varying a sampling rate while plotting the ECG signal in correlation to a size and pixel density of the display.

17. The method of claim 1, wherein the ECG signal includes a plurality of signal channels, and wherein the synchronization includes spacing apart each incoming signal channel along an axis corresponding to a polled height of the display and a number of the plurality of channels.

18. The method of claim 17, wherein the synchronization includes dynamically adjusting a baseline of the ECG signal being plotted along an axis and plotting the ECG signal on the formed grid of the display pixel by pixel for each channel.

19. The method of claim 1, wherein the synchronization includes,
plotting a first set of lines of a first color at a first interval and first thickness to form an X-axis, and
dynamically adjusting a baseline of the ECG signal being plotted along a Y-axis.

20. A system for automatically plotting electrocardiogram grid for a display device, said system comprising:
a display; and
a computer processor configured to:
divide axes of a grid into spatial intervals of grid lines to form a dynamically oriented background grid corresponding to the display, and
synchronize data points corresponding to a real time incoming electrocardiogram (ECG) signal with the background grid to plot the ECG signal on the background grid, wherein the dividing and the synchronizing dynamically repositioning each grid line and each incoming data point based on a configuration of the display.
